(19) United States
(12) Patent Application Publication
(10) Pub. No.: US 2017/0022286 A1
(43) Pub. Date: Jan. 26, 2017

(54) MEDICAL USES OF CRTAM AGONISTS

(71) Applicant: The Board of Trustees of the Leland Stanford Junior University, Stanford, CA (US)

(72) Inventor: Holbrook Kohrt, Palo Alto, CA (US)

(21) Appl. No.: 15/078,901

(22) Filed: Mar. 23, 2016

Related U.S. Application Data
(60) Provisional application No. 62/137,123, filed on Mar. 23, 2015.

(51) Int. Cl. C07K 16/28; C07K 16/32; C07K 16/30

(52) U.S. Cl. CPC C07K 16/2896; C07K 16/30; C07K 16/3061; C07K 16/32; C07K 2317/24; C07K 2317/75

(57) ABSTRACT

Methods and compositions relating to use of CRTAM agonists are provided. In some embodiments, the present invention provides methods and compositions relating to use of CRTAM in the treatment of cancer, including enhancing the efficacy of antibody therapy directed to cancer cells.

### CROSS-REFERENCE

This application claims the benefit of U.S. Provisional Patent Application No. 62/137,123, filed Mar. 23, 2015, which application is incorporated herein by reference in its entirety.

### BACKGROUND OF THE INVENTION

Tumors persist and grow in part because they suppress the endogenous immune response through a variety of mechanisms. Activating the immune system for therapeutic benefit in cancer has long been a goal in immunology and oncology. A number of approaches are in development to increase host anti-tumor responses. Points of intervention include enhancing phagocytosis of tumor cells, overcoming immunosuppression mechanisms of the tumor, and enhancing the activity of immune cells, such as T cells, phagocytes, and NK cells.

For example, tumors may prevent immunization, trigger the ‘wrong’ immune response or enable the local accumulation or expansion of T_{reg} cells that would oppose the activity of effector T cells. Indeed, infiltration of T_{reg} cells correlates with poor prognosis in a variety of epithelial tumour types. Tumours may downregulate their expression of MHC class I molecules or their expression of target tumour antigens and can also produce a variety of surface molecules (for example, PD-L1 or PD-L2) that engage receptors on the surfaces of activated T cells (PD-1), causing T-cell anergy or exhaustion. Tumor cells also expression surface molecules that inhibit phagocytosis. Expression of such suppressive ligands can be associated with oncogenic mutations seen in many cancers (for example, PTEN loss). Additionally, tumours can release immunosuppressive molecules, such as indoleamine 2,3-dioxygenase (IDO), which consumes tryptophan and limits T-cell function. Myeloid-derived suppressor cells can also be recruited into the tumour, and release additional T-cell suppressors, such as arginase and nitrous oxide synthase. Hypoxia in the tumour microenvironment may promote the generation of adenosine, which inhibits effector T-cell function in a similar way. Hypoxia can also lead to the production of CCL28, which attracts immigration of T_{reg} cells. Finally, tumour stroma cells can also suppress the function of effector lymphocytes. For example, mesenchymal stem cells block proliferation and function of effector T cells, whereas tumour vascular cells can suppress T-cell adhesion (to tumour endothelium) and prevent homing to tumours.

Cancer immunotherapy is starting to reveal significant and sometimes spectacular responses in several indications. Yet, even in diagnostically defined populations, these responses can be transient or require continued dosing. If therapeutic regimens can be matched to appropriate immunotherapies, activating a patient's immune system may be the best way to ensure that responses are converted to a long-term and durable benefit.

### SUMMARY OF THE INVENTION

The present invention demonstrates effective treatment of cancer by agonizing Cytotoxic and Regulatory T Cell Molecule (CRTAM) by administration of CRTAM agonist therapy. The present invention demonstrates that administration of a CRTAM agonist can enhance the ability of a subject's immune system to target and destroy cancer cells. In particular, the present invention demonstrates that agonizing CRTAM can increase effector cell killing of tumor cells when administered as a monotherapy, or in conjunction with a tumor-specific antibody. The present disclosure specifically demonstrates that administration of a CRTAM agonist can augment the ADCC capability of immune effector cells such as NK cells, including the response against cancer cells to which an antibody has bound.

In some embodiments, methods are provided for enhancing antibody-dependent cellular cytotoxicity (ADCC) of effector cells in a subject in need thereof, the method comprising administering to the subject a CRTAM agonist therapy, wherein the CRTAM agonist therapy comprises administration of one or more doses of a CRTAM agonist according to a regime correlated with elevated ADCC of the effector cells. The CRTAM can be used as a monotherapy in such methods, or alternatively is combined with a tumor-specific antibody.

In some embodiments CRTAM agonist therapy is combined with anti-tumor antibody therapy; including without limitation a serial, staged CRTAM agonist therapy relative to anti-tumor antibody therapy. The present invention demonstrates that CRTAM levels on immune effector cells are upregulated following contact with tumor cells bound by anti-tumor antibody. Administration of a CRTAM agonist following the increased expression on immune effector cells enhances killing of the tumor cells.

In one embodiment, the present invention provides methods of treating cancer by administering to a patient a composition comprising a CRTAM agonist, including without limitation an agonist antibody that specifically binds to and activates CRTAM. In some embodiments, such administration is to an individual who is receiving or has received anti-tumor antibody therapy. In some embodiments, the individual has received anti-tumor antibody therapy a period of time prior to the administering of the CRTAM agonist. In such embodiments, the period of time may be selected so that, prior to the administering of the CRTAM agonist, CRTAM expression has increased on surfaces of effector cells that mediate antibody-dependent cellular cytotoxicity (ADCC). Administration of such therapy results in increased ADCC of the targeted tumor cells. In some embodiments, apoptosis of the tumor cells upon administering of the CRTAM agonist is increased relative to that observed absent the CRTAM agonist. In some embodiments, tumor growth upon administering of the CRTAM agonist is reduced relative to that observed absent the CRTAM agonist.

In some embodiments, the present invention provides methods treating cancer by of administering CRTAM agonist therapy together with agonist therapy directed at one or more inducible immune effector cell surface markers other than CRTAM. In some embodiments, such methods can further involve administering anti-tumor antibody therapy. Thus, in some embodiments, the present invention provides methods of treating cancer that include steps of i) administering anti-tumor antibody therapy; ii) administering anti-CRTAM agonist therapy; and iii) administering agonist therapy targeting at least one inducible immune effector cell surface marker other than CRTAM. In some such embodiments, CRTAM agonist therapy is administered a first period of time after administration of anti-tumor antibody therapy. Agonist therapy targeting at least one inducible immune effector cell surface marker other than CRTAM may be administered a second period of time after administration of anti-tumor antibody therapy. The first and second time periods can be relative to the same dose of anti-tumor antibody therapy, where the first and second time periods are the same or different.

In some embodiments, methods of the invention involve determining CRTAM expression level on effector cells in a subject, e.g. prior to administering a CRTAM agonist for treatment of cancer. CRTAM expression level can be determined at multiple time points, e.g. before, substantially simultaneously with, and/or after administration of one or more doses of a CRTAM agonist. In some embodiments, CRTAM expression level is determined before, substantially simultaneously with, and/or after administration of one or more doses of an anti-tumor antibody therapy. In some embodiments, CRTAM expression level is determined after administration of anti-tumor antibody therapy and before administration of CRTAM agonist therapy. In some embodiments, anti-tumor antibody therapy is administered, followed by a delay that lasts a period of time, and then CRTAM agonist therapy is delivered, and CRTAM expression level is determined at least once, and optionally multiple times, within the period of time and/or prior to the anti-tumor antibody therapy. In some such embodiments, the period of time has a length determined by a pre-determined change in CRTAM expression level, e.g. a predetermined increase in CRTAM expression on immune effector cells. In some embodiments of methods of the present invention, expression levels both of CRTAM and of an inducible immune effector cell surface marker other than CRTAM are determined, at the same or different times. CRTAM expression level, and/or expression level of an inducible immune effector cell surface marker other than CRTAM can be determined by analysis of a patient sample, including without limitation blood samples and derivatives thereof, tissue samples, tumor biopsy samples, etc.

In some of the embodiments where the method of treating cancer further comprises one or more of the steps of determining CRTAM expression level on surfaces of the effector cells, the step of determining comprises detecting CRTAM protein. In some of the embodiments where the method of treating cancer further comprises one or more of the steps of determining CRTAM expression level on surfaces of the effector cells, the step of determining comprises detecting a surrogate marker for CRTAM expression on surfaces of the effector cells.

Pharmaceutical compositions comprising CRTAM agonist agents are provided. In some embodiments, a CRTAM agonist is or comprises a non-antibody agent. In some embodiments, such a non-antibody agent is or comprises an aptamer that specifically binds to CRTAM. In some embodiments, a CRTAM agonist is or comprises an antibody agent that specifically binds to CRTAM. In some embodiments, such an antibody agent is or comprises an intact antibody. In some embodiments, such an antibody agent is or comprises a monoclonal antibody (mAb). In some embodiments, such an antibody agent is or comprises a humanized or human antibody, or includes antigen binding elements of a human or humanized antibody. In some embodiments, such an antibody agent is a multi-specific agent, such as a bi-specific antibody. In some such embodiments, the multi-specific agent binds specifically to CRTAM and to an inducible immune effector cell surface marker other than CRTAM. In some embodiments, the multi-specific agent binds specifically to CRTAM and to a tumor antigen. In some embodiments, the multi-specific agent binds specifically to CRTAM and to another antigen, which other antigen is not a tumor antigen.

Kits, or articles of manufacture, or methods for the preparation of a medicament containing components relevant to administration of CRTAM agonist therapy and/or detection of CRTAM expression are also provided, which kits or articles of manufacture may comprise, without limitation, a pharmaceutical composition comprising a CRTAM agonist; compositions for determining expression of CRTAM; an antibody specific for a tumor antigen; and the like. The CRTAM agonist may be administered within a period of time subsequent to the administration of the anti-tumor antibody during which period of time the expression of CRTAM is increased on the surface of effector cells that mediate antibody-dependent cellular cytotoxicity (ADCC).

### BRIEF DESCRIPTION OF THE DRAWINGS

FIG. 1. CRTAM Induction on NK cells with mAb Therapy CRTAM expression on CD3⁻ CD56⁺ NK cells after 24 hours of culture with HER18 (HER2+ tumor cell) at a 1:1 cell ratio with or without IFNg (10 ng/mL), or anti-HER2 mAb (trastuzumab, 10 μg/mL) assayed by flow cytometric assessment of median flouresence intensity (MFI). NK cell stimulation by IFN-gamma co-culture or by FcR (CD16) activation induces upregulated expression of CRTAM on the NK cell.

FIG. 2. CRTAM Agonism Enhances NK Function In Vitro. Anti-CRTAM agonistic mAb increases trastuzumab-mediated NK cell cytotoxicity on tumor cells. To evaluate NK cell function, purified NK cells were isolated from three independent, healthy donor PBMCs and cultured for 24 hours together with trastuzumab (10 μg/mL) and irradiated (5,000 rads) HER2-expressing cancer cells at a ratio of 1:1. After 24 hours, NK cells were isolated by negative selection and assessed for purity (>90% purity as defined by CD3⁻CD56⁺ flow cytometry) and activation (>50% expression of CRTAM). HER2-expressing cancer cell lines including HER18, were cultured for 18 hours with pre-activated, purified NK cells in media alone, or with anti-CRTAM (10 μg/mL) alone, trastuzumab (10 μg/mL) alone, or trastuzumab plus anti-CRTAM mAbs (both at 10 μg/mL). Cells were washed and incubated with annexin V and 7-AAD to determine percent apoptotic tumor cells by annexin V and 7-AAD staining and chromium-labeled HER2-expressing cancer cell lines were cultured for 4 hours with preactivated, purified NK cells in media alone, or with anti-CRTAM mAb alone, trastuzumab (10 μg/mL) alone, or trastuzumab plus anti-CRTAM mAbs, as well as two arms with triplet therapy. Shown is percent lysis of target cells by chromium release at 10:1 effector:target ratio.

FIG. 3. CRTAM Agonism Enhances NK Function In Vitro. Anti-CRTAM agonistic mAb increases trastuzumab-mediated NK cell cytotoxicity on tumor cells. To evaluate NK cell function, purified NK cells were isolated from three independent, healthy donor PBMCs and cultured for 24 hours together with trastuzumab (10 μg/mL) and irradiated (5,000 rads) HER2-expressing cancer cells at a ratio of 1:1. After 24 hours, NK cells were isolated by negative selection and assessed for purity (>90% purity as defined by CD3⁻CD56⁺ flow cytometry) and activation (>50% expression of CRTAM). HER2-expressing cancer cell lines including HER18, were cultured for 18 hours with pre-activated, purified NK cells in media alone, or with anti-CRTAM (10 μg/mL) alone, trastuzumab (10 μg/mL) alone, trastuzumab plus anti-CRTAM mAbs (both at 10 μg/mL), or trastuzumab plus anti-CD137 mAbs (both at 10 μg/mL). Cells were washed and incubated with annexin V and 7-AAD to determine percent apoptotic tumor cells by annexin V and 7-AAD staining and chromium-labeled HER2-expressing cancer cell lines were cultured for 4 hours with preactivated, purified NK cells. Shown is percent lysis of target cells by chromium release at 10:1 effector:target ratio.

FIGS. 4A-4B. CRTAM Agonism Enhances NK Function In Vivo. Anti-CRTAM agonistic mAb enhances anti-breast cancer activity of trastuzumab in vivo against HER2-overexpressing breast cancer cell lines. nu/nu mice were inoculated with 5×10⁶ HER18 breast tumor cells s.c. on the left flank. After tumor inoculation, mice received either trastuzumab on day 7, anti-CRTAM on day 7, or anti-CD137 on day 7, trastuzumab on day 7 and anti-CD137 antibody on day 8, or trastuzumab on day 7 and anti-CD137 antibody on day 8 with each treatment repeated weekly for a total of 3 weeks. Mice (10 per group) were monitored for tumor growth and survival.

### DEFINITIONS

Below are provided certain definitions of terms used herein, many or most of which confirm common understanding of those skilled in the art.

Cytotoxic and Regulatory T Cell Molecule (CRTAM). The CRTAM gene is upregulated in CD4-positive and CD8-positive T cells and encodes a type I transmembrane protein with V and C1-like Ig domains). The Genbank reference sequence for the human gene has the accession number NM_019604. The 393-amino acid human protein contains a 17-amino acid leader sequence, followed by a 269-amino acid extracellular region, a 25-amino acid transmembrane domain, and an 82-amino acid cytoplasmic domain. The extracellular region has 5 potential N-glycosylation sites and 5 conserved cysteines, 4 of which appear to form 2 Ig-like domains. The cytoplasmic region has 3 conserved phosphorylation sites. Among human tissues, CRTAM was abundantly expressed in spleen, thymus, small intestine, and peripheral blood leukocytes, with lower levels in testis, ovary, and colon. A number of tumor cells have been found to express the protein, including breast cancer, cervical cancer, colorectal cancer, endometrial cancer, gliomas, liver cancer, lung cancer, lymphomas, head and neck cancer, melanoma, ovarian cancer, pancreatic cancer, prostate cancer, renal cancer, skin cancer, stomach cancer, testics cancer, thyroid cancer and urothelial cancer.

The nectin-like protein Necl-2 is a ligand for CRTAM. The interaction between CRTAM and Necl-2 has been demonstrated in binding and adhesion assays, as well as in functional experiments using CD8+ T cells and NK cells. Engagement of CRTAM on human activated NK cells by its ligand Necl-2 on the surface of tumor cells promotes NK cell cytotoxicity towards otherwise poorly immunogenic targets.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection).

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Agonist: As used herein, the term “agonist” refers to an agent whose presence or level correlates with increase in level and/or activity of another agent (i.e., the agonized agent). In general, an agonist may be or include an agent of any chemical class including, for example, small molecules, polypeptides, nucleic acids, carbohydrates, lipids, metals, and/or any other entity that shows the relevant activating activity. An agonist may be direct, in which case it exerts its influence directly upon its target, or indirect, in which case it exerts its influence by other than binding to its target; e.g., by interacting with a regulator of the target, so that level or activity of the target is altered.

In some embodiments the agonist is an activating antibody that specifically binds to CRTAM, particularly an antibody that binds to human CRTAM. Various antibodies with specificity for CRTAM are known in the art and commercially available, e.g. clone Cr24.1 (see Kent S, et al. 2005. Blood. 106:779); clone 21A9 (Genetex); clone 210213 (see Llinas et al. Immunol. Lett., 2011; 134(2):113-21).

Alternative agonist may comprise, for example, sequences obtained from the CRTAM ligand NECL-2, e.g. a soluble portion of the protein, such as the extracellular domain. See, for example, Galibert et al. (2005) J. Biol. Chem. 280:21955-21964; Zhang et al. (2013) Structure 21(8):1430-9; and Martinet et al. (2015) Nat. Rev. Immunol. 15(4):243-54, each herein specifically incorporated by reference.

Agonist Therapy: The term “agonist therapy”, as used herein, refers to administration of an agonist that agonizes a particular target of interest to achieve a desired therapeutic effect. In some embodiments, agonist therapy involves administering a single dose of an agonist. In some embodiments, agonist therapy involves administering multiple doses of an agonist. In some embodiments, agonist therapy involves administering an agonist according to a dosing regimen known or expected to achieve the therapeutic effect, for example, because such result has been established to a designated degree of statistical confidence, e.g., through administration to a relevant population.

Antagonist: As used herein, the term “antagonist” refers to an agent whose presence or level correlates with decreased level or activity of another agent (i.e., the antagonized agent, or target. In general, an antagonist may be or include an agent of any chemical class including, for example, small molecules, polypeptides, nucleic acids, carbohydrates, lipids, metals, and/or any other entity that shows the relevant inhibitory activity. An antagonist may be direct (in which case it exerts its influence directly upon its target) or indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD, usually tetrameric, agents comprised of two identical heavy chain polypeptides and two identical light chain polypeptides. Each heavy chain is comprised of at least four domains, an amino-terminal variable (VH) domain, followed by three constant domains. A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains, an amino-terminal variable (VL) domain, followed by a carboxy-terminal constant (CL) domain, separated from one another by another “switch”.

Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced, or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art.

Moreover, the term “antibody” as used herein, can refer to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload such as a detectable moiety, a therapeutic moiety, a catalytic moiety, etc., or other pendant group,e.g., poly-ethylene glycol, etc.

Antibody Agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to, antibody constructs as described above. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins.

In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, ADCC is typically understood to involve Fc receptor (FcR)-bearing effector cells recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody dependent Cellular Phagocytosis. ACDP refers to a efers to a phenomenon in which target cells bound by antibody are phagocytosed by immune effector cells. Without wishing to be bound by any particular theory, ADCP is typically understood to involve phagocytic effector cells recognizing and subsequently killing antibody-coated target cells (e.g., cells that express on their surface specific antigens to which an antibody is bound).

Antigen: The term “antigen”, as used herein, refers to an agent that elicits an immune response; and/or an agent that binds to a T cell receptor or to an antibody. In some embodiments, and antigen binds to an antibody and may or may not induce a particular physiological response in an organism.

Biological Sample: As used herein, the term “biological sample” typically refers to a sample obtained or derived from a biological source (e.g., a tissue or organism or cell culture) of interest, as described herein. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc.

Biomarker: The term “biomarker” is used herein, consistent with its use in the art, to refer to a to an entity whose presence, level, or form, correlates with a particular biological event or state of interest, so that it is considered to be a “marker” of that event or state. A biomarker may be or comprises a marker for a particular disease state, or for likelihood that a particular disease, disorder or condition may develop. In some embodiments, a biomarker may be or comprise a marker for a particular disease or therapeutic outcome, or likelihood thereof.

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for treatment in the present application include precancerous (e.g., benign), malignant, pre-metastatic, metastatic, and non-metastatic cells. Cancers of interest include, without limitation, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

A cancer may be in the form of a solid tumor, which refers to an abnormal mass of tissue that usually does not contain cysts or liquid areas. Solid tumors may be benign or malignant. Different types of solid tumors are named for the type of cells that form them. Examples of solid tumors are sarcomas, carcinomas, lymphomas, mesothelioma, neuroblastoma, retinoblastoma, etc.

Combination Therapy: As used herein, the term “combination therapy” refers to those situations in which a subject is concomitantly exposed to two or more therapeutic regimens, e.g., two or more therapeutic agents. In some embodiments, two or more agents may be administered simultaneously; in some embodiments, such agents may be administered sequentially; in some embodiments, such agents are administered in overlapping dosing regimens.

Composition: A “composition” or a “pharmaceutical composition” according to this invention refers to the combination of two or more agents as described herein for co-administration or administration as part of the same regimen. It is not required in all embodiments that the combination of agents result in physical admixture, that is, administration as separate co-agents each of the components of the composition is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Inducible Effector Cell Surface Marker: As used herein, the term “inducible effector cell surface marker” refers to an entity, that typically is or includes at least one polypeptide, expressed on the surface of immune effector cells, including without limitation natural killer (NK) cells, which expression is induced or significantly upregulated during activation of the effector cells. In some embodiments, increased surface expression involves increased localization of the marker on the cell surface (e.g., relative to in the cytoplasm or in secreted form, etc). Alternatively or additionally, in some embodiments, increased surface expression involves increased production of the marker by the cell. In some embodiments, increased surface expression of a particular inducible effector cell surface marker correlates with and/or participates in increased activity by the effector cell. In some embodiments, an inducible effector cell surface marker is selected from a group consisting of a member of the TNFR family, a member of the CD28 family, a cell adhesion molecule, a vascular adhesion molecule, a G protein regulator, an immune cell activating protein, a recruiting chemokine/cytokine, a receptor for a recruiting chemokine/cytokine, an ectoenzyme, a member of the immunoglobulin superfamily, a lysosomal associated membrane protein. Certain exemplary inducible cell surface markers include, without limitation, CRTAM, CD38, CD137, OX40, GITR, CD30, ICOS, etc. In some particular embodiments, the term refers to any of the above-mentioned inducible cell surface markers other than CRTAM.

Patient: As used herein, the term patient, or subject, refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition, e.g., cancer. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF THE EMBODIMENTS

CRTAM expression on immune effector cells, e.g., NK cells, provides a therapeutic target for cancer therapy, where killing of cancer cells in a patient is enhanced by administering an agonist of CRTAM, which may be an agonist anti-CRTAM antibody, a soluble binding portion of NECL2, and the like. Administration of an effective dose of a CRTAM agonist increases the activity of immune effector cells and increases killing of tumor cells. Further, CRTAM on surfaces of immune effector cells (e.g., NK cells) can be induced upon contact with antibody-coated tumor cells in a FcR-dependent manner. In some embodiments, therefore, the present invention provides cancer therapies that involve administering at least an agonist of CRTAM. Optionally a combination therapy is provided, further comprising administration of an antibody that specifically binds to the targeted tumor cell. In some particular embodiments, the CRTAM agonist therapy is administered a period of time after the anti-tumor antibody therapy. In some embodiments, the therapy is a serial, staged therapy comprising administering an agonist of CRTAM a period of time after administration of an anti-tumor antibody therapy.

In some embodiments, therefore, ADCC-mediated cancer cell killing, and consequently reduction of tumor size and/or other cancer effects, is achieved by administering sequentially two distinct agents: first an antibody agent, which antibody agent includes an immunoglobulin Fc portion directed against one or more cancer antigens, and after a period of time, an agonist of CRTAM, e.g. after about 1 day, after about 2 days, after about 3 days, after about 4 days, after about 5 days, after about 6 days, after about 7 days, after about 8 days, after about 9 days, after about 10 days, after about 2 weeks, etc. The administration of the anti-cancer antibody agent leads to up-regulation of CRTAM on the surface of NK cells in the cancer patients, for example as determined in biological sample obtained from them, where an increase in expression may be at least about 10%, at least about 20%, at least about 30%, at least about 50%, at least about 75%, at least about 100%, or more. Consequent administration of a CRTAM agonist enhances activity of the effector cells.

This approach is shown to improve the clinical efficacy of antibodies directed against cancer-specific antigens that are already used for treating patients or that are still under development. It may be further combined with a standard of care therapy for a cancer, or, if appropriate, this approach may be applied to a cancer patient who has failed standard of care therapy by enhancing and/or prolonging ADCC response against cancer cells and consequently the therapeutic effect of the antibody directed against a cancer antigen.

The CRTAM agonist, alone or in combination with a tumor specific antibody, is provided in a therapeutically effective dose. The dose is sufficient to increase killing of tumor cells in the treated individual. Examples of indicia of effectiveness include, without limitation, a decrease in tumor volume of at least about 5%, 10%, at least about 15%, at least about 20%, at least about 25%, at least about 35%, at least about 45%, at least about 50%, at least about 75%, at least about 85%, at least about 90%, at least about 95%, or more relative to the tumor size in the absence of treatment. Alternatively an effective dose may be a dose that stabilizes tumor size over a period of time, i.e. with no increase in size for at least about one week, two weeks, three weeks, one month, two months, three months or more. An effective dose may be monitored in terms of increase of killing of cancer cells in an in vitro or in vivo assay, where killing is increased at least about 5%, 10%, at least about 15%, at least about 20%, at least about 25%, at least about 35%, at least about 45%, at least about 50%, at least about 75%, at least about 85%, at least about 90%, at least about 95%, or more relative to the killing in the absence of the CRTAM agonist treatment.

Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Technologies provided herein are useful in the treatment of any tumor, as described herein, including without limitation hematologic malignancies, such acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms; carcinomas; lymphomas, melanomas, sarcomas, gliomas, mesotheliomas, etc. In some embodiments, a tumor is characterized by no or low expression of CRTAM on the surface of tumor cells. In some embodiments, a tumor is characterized by significant expression of CRTAM on the surface of tumor cells; in some such embodiments, tumor cells express CRTAM on their surfaces at levels significantly higher than non-tumor cells. In some particular embodiments, a tumor is an advanced tumor, and/or a refractory tumor. In some embodiments, a tumor is characterized as advanced when cancer patients with such tumor are not candidates for conventional chemotherapy.

### CRTAM Agonists

Some agonists of CRTAM are known in the art. Others can be identified, generated, and/or characterized as described herein. In some embodiments, a CRTAM agonist is or comprises an antibody agent (e.g., an intact antibody) specific for human CRTAM. Antibodies that recognize the extracellular domain of CRTAM (and in particular of human CRTAM) have been generated using different approaches but those appropriate for enhancing the ADCC and cancer cell killing are those that both bind to, and activate CRTAM-mediated signaling.

An antibody agent that agonizes CRTAM may be or comprise an intact antibody, or another antibody format, e.g., as known in the art and/or described herein, including for example a single chain format or a multi-specific format. An antibody that agonizes CRTAM may be polyclonal or, preferably, monoclonal and/or may be of non-human origin (e.g., of rodent or camel origin) or, preferably, may be chimeric, humanized or human. In some particular embodiments, such a multi-specific agent binds specifically to CRTAM and to an inducible immune effector cell surface marker other than CRTAM. In other embodiments, the multi-specific agent binds specifically to CRTAM and to a tumor antigen. In other embodiments, the multi-specific agent binds specifically to CRTAM and to another antigen, which other antigen is not a tumor antigen.

In some other embodiments, a CRTAM agonist is or comprises a non-antibody agent. In some embodiments, such a non-antibody-agent CRTAM agonist is or comprises a nucleic acid, saccharide, lipid, small molecule, metal, or a combination thereof. In some embodiments, a non-antibody-agent CRTAM agonist is an aptamer that specifically binds to CRTAM.

The nectin-like protein Necl-2 is a ligand for CRTAM. In some embodiments a CRTAM agonist can be or comprises the whole or fragments or other variants of the extracellular domain of CD31, particularly soluble fragments that lack the transmembrane domain of Necl-2.

### Anti-Tumor Antibody Therapy

Antibody agents are known in the art that have been designed or selected to bind to tumor cell antigens in order to kill tumor cells by a) delivering a toxic payload associated with the antibody; b) blocking activity of a tumor cell surface receptor that is thought to be involved in cell proliferation and/or survival; c) agonizing activity of a tumor cell surface receptor that is thought to be involved in triggering apoptosis or cell death; and/or d) displaying bound antibody on tumor cell surface, so that immune mechanisms like complement-dependent cytotoxicity (CDC) and/or antibody-dependent cell toxicity (ADCC) are triggered and directed at the tumor. Anti-tumor antibody agents clinically available for a number of different targets.

Antibodies for use as an anti-tumor agent in combination with a CRTAM agonist include, without limitation, those set forth in the table below.

| | Antibody (commercial or | |
|---|---|---|
| Human Antigen | scientific name) | Cancer indication |
| CD2 | Siplizumab | Non-Hodgkin's Lymphoma |
| CD3 | UCHT1 | Peripheral or Cutaneous T-cell |
| CD4 | HuMax-CD4 | Lymphoma |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic |
| | Ibritumomab, Obinutuzumab, | leukemia) |
| CD22 (SIGLEC2) | Inotuzumab, tetraxetan, CAT- | Chemotherapy-resistant hairy cell |
| | 8015, DCDT2980S, | leukemia, Hodgkin's lymphoma |
| | Bectumomab | |
| CD30 | Brentuximab vedotin | |
| CD33 | Gemtuzumab ozogamicin | Acute myeloid leukemia |
| | (Mylotarg) | |
| CD37 | TRU-016 | Chronic lymphocytic leukemia |
| CRTAM | Daratumumab | Multiple myeloma, hematological |
| | | tumors |
| CD40 | Lucatumumab | Non-Hodgkin's lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | Colon, lung and pancreas tumors and |
| CD262 (TRAILR2) | HGS-ETR2, CS-1008 | haematological malignancies |
| CD326 (Epcam) | Edrecolomab, 17-1A, IGN101, | Colon and rectal cancer, malignant |
| | Catumaxomab, | ascites, epithelial tumors (breast, colon, |
| | Adecatumumab | lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, | Breast cancer |
| | Ado-trastuzumab emtansine | |
| CAIX (CA9) | cG250 | Renal cell carcinoma |
| EGFR (c-erbB) | Cetuximab, Panitumumab, | Solid tumors including glioma, lung, |
| | nimotuzumab and 806 | breast, colon, and head and neck |
| | | tumors |
| EPHA3 (HEK) | KB004, IIIA4 | Lung, kidney and colon tumors, |
| | | melanoma, glioma and haematological |
| | | malignancies |
| Episialin | Epitumomab | Epithelial ovarian tumors |
| FAP | Sibrotuzumab and F19 | Colon, breast, lung, pancreas, and head |
| | | and neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and |
| | | prostate tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, | Breast, ovary and lung tumors |
| | SCH900105 | |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumour vasculature |

Any such anti-tumor antibody agents can be utilized in combination with CRTAM agonist therapy in the practice of the present invention. In some embodiments, when effector cells are exposed to tumor cells bound by the anti-tumor antibody agents, CRTAM expression on the surface of such effector cells is increased as compared with that observed absent such exposure. In some embodiments, when effector cells are exposed to tumor cells bound by the anti-tumor antibody agents, the expression of a second inducible effector cell surface marker (e.g., CD38, CD137, OX40, GITR, ICOS, CD30, etc.) is also increased on the surface of such effector cells as compared with that observed absent such exposure.

In some embodiments, when a CRTAM agonist is administered to a subject a period of time after the subject receives the anti-tumor antibody agent, ADCC against tumor cells is enhanced as compared with that observed absent the administering of the CRTAM agonist, e.g. enhanced at least about 10%, at least about 25%, at least about 50%, at least about 75%, at least about 100% or more.

Anti-tumor antibodies of particular use in the methods of the invention include, without limitation, antibodies specific for CD20, such as Rituximab, Tositumomab, or Ibritumomab; antibodies specific for CD52, such as Alemtuzumab; antibodies specific for HER2, such as Trastuzumab; antibodies specific for EGFR, such as Cetuximab; antibodies specific for CD326, such as Edrecolomab; antibodies specific for CD38, such as Daratumumab. Selection of a suitable antibody may be identified or characterized via one or more ex vivo, in vivo, or in vitro techniques, as will be familiar to those of skill in the art, reading the present specification.

In some particular embodiments, cancer-associated antigens are identified and/or characterized by genomic profiling, e.g., to identify genes for which one or more features of the gene's expression correlates with one or more features of tumor character and/or that identifies genes encoding proteins likely to be partially or wholly displayed on tumor cell surfaces. Alternatively or additionally, in some embodiments, one or more useful cancer-associated antigens may be identified and/or characterized using a technique such as magnetic separation with antibody-coated magnetic beads, “panning” with antibody attached to a solid matrix, mass spectrometry, flow cytometry, proteomic profiling, immunohistochemistry of biopsy samples, and combinations thereof.

### Inducible Effector Cell Surface Markers

In some embodiments of the invention, an inducible effector cell surface marker other than CRTAM is targeted, in combination with CRTAM and optionally further in combination with an anti-tumor antibody. Such inducible surface markers are known to those of skill in the art, and include, without limitation, certain members of the TNFR family, certain members of the CD28 family, certain cell adhesion molecules, certain vascular adhesion molecules, certain G protein regulators, certain immune cell activating proteins, certain recruiting chemokine/cytokines, certain receptors for recruiting chemokine/cytokines, certain ectoenzymes, certain members of the immunoglobulin superfamily, certain lysosomal associated membrane proteins, and combinations thereof. In some embodiments, inducible effector cell surface markers other than CRTAM are selected from CD38, CD137, OX40, GITR, CD30, ICOS, etc.

In some embodiments of the present invention, in addition to a CRTAM agonist, a second agonist of an inducible effector cell surface marker is administered to cancer patient who has previously received antitumor antibody therapy to improve the ADCC-mediated anti-tumor therapeutic effect of the antitumor antibody. Many potentially useful agonists of the inducible effector cell surface markers are known in the art. Others can be identified, generated, and/or characterized, for example as described in US published Patent Application 20120321646 A1; or in WO2015/009726, each specifically incorporated by reference.

In some embodiments, an agonist of an inducible effector cell surface marker for use in accordance with the present invention is or comprises a physiological ligand of the inducible marker, or a fragment or variant thereof. In other embodiments the agonist is an antibody or effective fragment or variant thereof. In general, an antibody agent that agonizes an inducible effector cell surface marker may be or comprise an intact antibody, or another antibody format (e.g., as known in the art and/or described herein), including for example a single chain format or a multi-specific format. In some particular embodiments, an antibody agent that agonizes an inducible effector cell surface marker is provided and/or utilized in a multi-specific (e.g. bi-specific) format that also targets CRTAM.

### Combination Therapies

CRTAM agonistic therapies may be combined with other anti-cancer therapies, including for example administration of chemotherapeutic agents, other immunomodulatory agents (including other agonists and/or antagonists of other inducible effector cell surface markers), radiation therapy, high-frequency ultrasound therapy, surgery, etc. In some embodiments the combination shows a synergistic effect in treating cancer.

For example, as described herein, in some embodiments, CRTAM agonist therapy is combined with anti-tumor antibody therapy. Alternatively or additionally, in some embodiments, CRTAM agonist therapy is combined with agonist therapy targeting an inducible effector cell surface marker other than CRTAM and/or with any other compound or treatment known to show therapeutic efficacy in treating cancer. One or more doses of agents administered in combination may be administered at the same time; in some such embodiments, agents may be administered in the same composition. More commonly, however, agents are administered in different compositions and/or at different times. To give a specific example, as described herein, in many embodiments, agonist therapy that targets CRTAM is administered in combination with anti-tumor antibody therapy, and desirably a period of time after administration of such anti-tumor antibody therapy.

In some particular such embodiments, the relevant period of time is sufficient for increased surface expression CRTAM to a level that is at least about 10%, 20%, 50%, 100%, 150%, 200% or more than that observed on the relevant effector cells (e.g., NK cells) prior to the administration of the anti-tumor antibody therapy. In some embodiments, surface expression level of the inducible effector cell surface marker is monitored between administration of the anti-tumor antibody therapy and the agonist therapy, for example at one or more specified time points, e.g., at time points such as about 1 hour, 3 hours, 6 hours, 12 hours, and/or 24 hours, or more. In some embodiments, effector cell surface marker expression is monitored in assays that utilize human cancer cells, tissues, and/or other biological materials. In some embodiments, the agonist therapy is not administered until a desired level of increased surface expression is achieved. In some embodiments, level of CRTAM expression is determined via detection of a surrogate marker, e.g., an alternative marker of effector cell activation, rather than of CRTAM itself.

In some particular embodiments, administration of agonist therapy targeting CRTAM may occur a period of time that starts at least 1 hour, 3 hours, 6 hours, 12 hours, 24 hours, 72 hours, or up to 5 days or more after the administration of anti-tumor antibody therapy. In some embodiments, administration of agonist therapy occurs within a time period during which CRTAM is expressed at an elevated level on surfaces of effector cells. In some embodiments, such a time period begins within an hour or so of administration of anti-tumor antibody therapy and lasts at least about 2, about 5, about 11, about 23 hours, about 71 hours or more. In some embodiments, such a time period lasts between about 1 hour (or less than hour) and about 24 or more hours or about 72 or more hours. In some embodiments, such a time period begins within about 1 hour, about 3 hours, about 6 hours, or about 12 hours of administration of anti-tumor antibody therapy; in some embodiments, such a time period lasts until at least about 12 hours, about 24 hours, about 72 hours, or about 5 days or more after administration of anti-tumor antibody therapy. In some embodiments, such a time period does not last more than about 5 days, about 72 hours, or about 24 hours after administration of anti-tumor antibody.

Other known compounds or treatments that show therapeutic efficacy in treating cancer may include, for example, one or more alkylating agents, anti-metabolites, anti-microtubule agents, topoisomerase inhibitors, cytotoxic antibiotics, angiogenesis inhibitors, immunomodulators, vaccines, cell-based therapies (e.g. allogeneic or autologous stem cell transplantation), organ transplantation, radiation therapy, surgery, etc.

Still further, in some embodiments, CRTAM agonist therapy may be combined with one or more palliative, e.g., pain relieving, anti-nausea, anti-emesis, etc. therapies, particularly when relieves one or more symptoms known to be associated with the relevant cancer, or with another disease, disorder or condition to which a particular cancer patient is susceptible or from which the particular cancer patient is suffering.

### Dosing and Administration

Pharmaceutical compositions comprising a CRTAM agonist, an anti-tumor antibody and/or any other therapeutically active agent for use in accordance with the present invention may be prepared for storage and/or delivery using any of a variety of techniques and/or technologies known and/or available to those skilled in the art.

In some embodiments, a therapeutically active agent utilized in accordance with the present invention is administered according to a dosing regimen approved by a regulatory authority such as the United States Food and Drug Administration (FDA) and/or the European Medicines Agency (EMEA), e.g., for the relevant indication. Those skilled in the art will be aware, or will readily be able to determine, approved dosing regimens for a variety of agents, including for example, a variety of anti-tumor antibodies.

Those skilled in the art, reading the present disclosure will appreciate various modifications of dosing regimens, etc, that are within the scope of the present invention. For example, CRTAM agonist therapy can be utilized as a monotherapy. In other embodiments, CRTAM agonist therapy is combined with other anti-cancer therapies, and particularly with anti-tumor antibody therapy. In some embodiments, one or more doses of CRTAM agonist is administered substantially simultaneously with a dose of anti-tumor antibody; in some embodiments, one or more doses of CRTAM agonist is administered after a delay relative to a particular dose of anti-tumor antibody; in some embodiments, doses of CRTAM agonist are administered after a delay relative to each dose of an anti-tumor antibody. Alternatively or additionally, in some embodiments, CRTAM agonist therapy is administered in accordance with the present invention together with lower or less frequent doses of anti-tumor antibody therapy than are standard (e.g., approved) when the anti-tumor antibody therapy is used as monotherapy (or otherwise absent the CRTAM agonist therapy). In some such embodiments, addition of yet another anti-cancer therapy may be particularly useful.

In some embodiments, it is desirable to tailor dosing regimens, and particularly to design sequential dosing regimens, based on timing and/or threshold expression levels of inducible markers (including CRTAM), whether for particular types of tumors, particular tumors, particular patient populations (e.g., carrying genetic markers), and/or particular patients. In some such embodiments, therapeutic dosing regimens may be combined with or adjusted in light of detection methods that assess expression of one or more inducible markers prior to and/or during therapy.

In some embodiments, dosing and administration according to the present invention utilizes active agent having a desired degree of purity combined with one or more physiologically acceptable carriers, excipients or stabilizers in any or variety of forms. These include, for example, liquid, semi-solid and solid dosage forms, such as liquid solutions (e.g., injectable and infusible solutions), dispersions or suspensions, tablets, pills, powders, liposomes and suppositories. In some embodiments, a preferred form may depend on the intended mode of administration and/or therapeutic application. Typical preferred compositions are in the form of injectable or infusible solutions, such as compositions similar to those used for passive immunization of humans with other antibodies.

Ingredient(s) can be prepared with carriers that protect the agent(s) against rapid release and/or degradation, such as a controlled release formulation, including implants, transdermal patches, and microencapsulated delivery systems. Biodegradable, biocompatible polymers can be used, such as polyanhydrides, polyglycolic acid, polyorthoesters, and polylactic acid.

In general, each active agent is formulated, dosed, and administered in therapeutically effective amount using pharmaceutical compositions and dosing regimens that are consistently with good medical practice and appropriate for the relevant agent(s) (e.g., for agents such as antibodies). Pharmaceutical compositions containing active agents can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transfermal, or other kinds of administration involving physical breaching of a tissue of a subject and administration of the pharmaceutical composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular cancer being treated (e.g., type, stage, location, etc), the clinical condition of a subject (e.g., age, overall health, etc), the site of delivery of the agent, the nature of the agent (e.g. an antibody or other protein-based compound), the mode and/or route of administration of the agent, the presence or absence of combination therapy, and other factors known to medical practitioners.

In some embodiments, one or more features of a particular pharmaceutical composition and/or of a utilized dosing regimen may be modified over time (e.g., increasing or decreasing amount of active in any individual dose, increasing or decreasing time intervals between doses, etc), for example in order to optimize a desired therapeutic effect or response (e.g., an ADCC response).

In general, type, amount, and frequency of dosing of active agents in accordance with the present invention in governed by safety and efficacy requirements that apply when relevant agent(s) is/are administered to a mammal, preferably a human. In general, such features of dosing are selected to provide a particular, and typically detectable, therapeutic response as compared with what is observed absent therapy. In context of the present invention, an exemplary desirable therapeutic response may involve, but is not limited to, inhibition of and/or decreased tumor growth, tumor size, metastasis, one or more of the symptoms and side effects that are associated with the tumor, as well as increased apoptosis of cancer cells, therapeutically relevant decrease or increase of one or more cell marker or circulating markers and the like. Such criteria can be readily assessed by any of a variety of immunological, cytological, and other methods that are disclosed in the literature. In particular, the therapeutically effective amount of CRTAM agonist, alone or in combination with an third agent, can be determined as being sufficient to enhance ADCC killing of cancer cells targeted by the first agent.

As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population.

The term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

An effective amount of an active agent or composition comprising it can be readily using techniques available in the art including, for example, considering one or more factors such as the disease or condition being treated, the stage of the disease, the age and health and physical condition of the mammal being treated, the severity of the disease, the particular compound being administered, and the like.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of the first agent, second agent, and/or the third agent at increasing doses. Consequently, the relative amounts of the each agent within a pharmaceutical composition may also vary, for example, each composition may comprise between 0.001% and 100% (w/w) of the corresponding agent.

Therapeutic compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The formulation of each agent should desirably be sterile, as can be accomplished by filtration through sterile filtration membranes, and then packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent, such as water or 1,3 butanediol, for example. Other parentally-administrable formulations which are useful include those which comprise the active ingredient in microcrystalline form, in a liposomal preparation, or as a component of biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. A non-exhaustive list of such additional pharmaceutically acceptable compounds includes buffers such as phosphate, citrate, and other organic acids; antioxidants including ascorbic acid and methionine; salts containing pharmacologically acceptable anions (such as acetate, benzoate, bicarbonate, bisulfate, isothionate, lactate, lactobionate, laurate, malate, maleate, salicylate, stearate, subacetate, succinate, tannate, tartrate, teoclate, tosylate, thiethiodode, and valerate salts); preservatives (such as octadecyidimethylbenzyl ammonium chloride; hexamethonium chloride; benzalkonium chloride, benzethonium chloride; sodium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or antibodies; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, one or more active agents utilized in practice of the present invention is administered according to an intermittent dosing regimen comprising at least two cycles. Where two or more agents are administered in combination, and each by such an intermittent, cycling, regimen, individual doses of different agents may be interdigitated with one another. In some embodiments, one or more doses of the second agent is administered a period of time after a dose of the first agent. In some embodiments, each dose of the second agent is administered a period of time after a dose of the first agent. In some embodiments, each dose of the first agent is followed after a period of time by a dose of the second agent. In some embodiments, two or more doses of the first agent are administered between at least one pair of doses of the second agent; in some embodiments, two or more doses of the second agent are administered between al least one pair of doses of the first agent. In some embodiments, different doses of the same agent are separated by a common interval of time; in some embodiments, the interval of time between different doses of the same agent varies. In some embodiments, different doses of the different agents are separated from one another by a common interval of time; in some embodiments, different doses of the different agents are separated from one another by different intervals of time.

If the first resting period's length is determined by existence or development of a particular biological or therapeutic event (e.g., induction of increased surface expression of an inducible effector cell surface marker), then the second resting period's length may be determined on the basis of different factors, separately or in combination. Exemplary such factors may include type and/or stage of a cancer against which anti-tumor antibody therapy (e.g., the first agent) is administered; identity and/or nature of a targeted tumor antigen, identity and/or properties (e.g., pharmacokinetic properties) of the first agent (e.g., of an anti-tumor antibody), and/or one or more features of the patient's response to therapy with the first agent. In some embodiments, length of one or both resting periods may be adjusted in light of pharmacokinetic properties (e.g., as assessed via plasma concentration levels) of one or the other of the administered agents. For example, a relevant resting period might be deemed to be completed with plasma concentration of the relevant agent is below about 1 μg/ml, 0.1 μg/ml, 0.01 μg/ml or 0.001 μg/ml, optionally upon evaluation or other consideration of one or more features of the patient's response (e.g., of degree of cancer reduction and/or magnitude and/or type of induced cancer-specific immune response).

In some embodiments, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc may be different for one or more cycles as compared with one or more other cycles. Ultimately, patient response is paramount.

### Articles of Manufacture and Kits

In another embodiment of the invention, each of first agent, the second agent, and, optionally, the third agent is provided in a separate article of manufacture. In particular, the second and third agent may target a further antigen on NK cells (such as CD137 and OX40), or further cancer-specific compound selected among chemotherapeutic compounds, cancer vaccines, signal transduction inhibitors, antibodies or other ligands that inhibit tumor growth, and immunomodulatory agents, among many others listed above as a potential third agent.

In another embodiment of the invention, an article of manufacture containing the first agent, the second agent, or, when appropriate, a third agent as described above is provided as a container with a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). For example, the formulation is packaged in clear glass vials with a rubber stopper and an aluminum seal. The label on, or associated with, the container indicates that the composition is used for treating the condition of choice.

The article of manufacture may further comprise a separate container comprising a pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use. If the second agent and the third agent are simultaneously, the article of manufacture may contain the second agent and the third agent in a single container, or appropriate materials and instructions for reconstituting the second agent and third agent in a single formulation may be provided. For example, the article of manufacture may allow providing each or the agent in an intravenous formulation as a sterile aqueous solution containing a total of 2 mg, 5 mg, 10 mg, 20 mg, 50 mg 100 mg, or more that are formulated, with appropriate diluents and buffers, at a final concentration of 0.1 mg/ml, 1 mg/ml, 10 mg/ml, or at an higher concentration.

Each of the first agent, second agent, and (when applicable) third agent can be provided within the kits-of-parts in the form of lyophilized is to be reconstituted with any appropriate aqueous solution that provided or not with the kits, or other types of dosage unit using any compatible pharmaceutical carrier. As the article of manufacture, this kits-of-parts is labeled for the treatment of a cancer and it may also contain a third agent, as defined above as a further, separate article of manufacture or within the article of manufacture containing the second agent. One or more unit dosage forms of the each of the first agent, the second agent, and, optionally, the third agent may be provided in a pack or dispenser device. Such a pack or device may, for example, comprise metal or plastic foil, such as a blister pack. The kit-of-parts may further comprise materials and/or devices suitable for measuring expression of the target of the first agent, CRTAM, the target of the third agent, and/or of a surrogate marker, on NK cells (e.g. a detectable labeled reagent that specifically binds to CRTAM, the target of the third agent, and/or of a surrogate marker, and references for expression). In order to use correctly such kits-of-parts, it may further comprise buffers, diluents, filters, needles, syringes, and package inserts with instructions for use in the treatment of cancer.

The instructions that are associated to the article of manufacture and/or the kits of the invention may be in the form of a label, a leaflet, a publication, a recording, a diagram, or any other means that can be used to inform about the correct use and/or monitoring of the possible effects of the agents, formulations, and other materials in the article of manufacture and/or in the kit. Instructions may be provided together with the article of manufacture and/or in the kit or may be provided separately but with the indication that indications are to be used in association with them.

### EXAMPLES

The invention is further described in detail by reference to the following experimental examples. These examples are provided for purposes of illustration only, and are not intended to be limiting. Thus, the invention should be construed to encompass any and all variations to the following examples which become evident as a result of the teaching provided herein. It is also to be understood that the terminology used herein is for the purpose of describing particular embodiments only, and is not intended to be limiting.

### Materials & Methods

Cell lines and culture. The human breast cancer cell lines BT474M1 (ATCC® HTB-20™) and MCF-7/HER2-18 (MCF-7 cells stably overexpressing HER2, also known as HER18; Benz CC et al.,1992.) were kindly provided as a gift from Byron Hann at UCSF (San Francisco, Calif., USA). The murine CD20-positive cell line, A20, and human CD20-positive B cell line, Raji, was purchased from ATCC. The BT474M1 cell line was cultured in DMEM medium, the Raji cell line in RPMI medium, and the MCF-7/HER2-18 cell line in DMEM/F12 1:1 medium. All media were purchased from Life Technologies. Cells were grown as adherent cultures at 37° C. in 5% CO₂ and passaged after detachment by 0.05% trypsin (Life Technologies). BT474M1 and MCF-7/HER-18 cells express HER2 with specific fluorescence indices (tumor MFI/isotype MFI) of 1.24 and 1.54, respectively. No detectable surface levels of CRTAM are observed on these cell lines by flow cytometry assessment.

Mice. Five- to six-week-old female athymic (nu/nu) nude Foxn1^{nu} and SCID mice (Prkdc^{scid}) were purchased from Harlan and Jackson Laboratories and were housed at the Laboratory Animal Facility at the Stanford University Medical Center.

Antibodies. Control rat IgG was purchased from Sigma-Aldrich. Human anti-human agonist CD137 monoclonal antibody (BMS-663513, IgG4) was provided by Bristol-Myers Squibb through a Material Transfer Agreement. Rituximab (murine-human chimeric anti-CD20, IgG1), Trastuzumab (humanized anti-human HER2/neu receptor, IgG1), and Trastuzumab D265A (a variant of Trastuzumab with a single alanine substitution at position 265; Clynes R et al., 2000, Nat Med 6:443) were obtained from Genentech through a Material Transfer Agreement.

Flow cytometry. Monoclonal antibodies against human antigens were used for staining of human PBMCs or purified NK cells. Stained cells were collected on a FACSCalibur or a LSRII 3-laser cytometer (BD Biosciences), and data were analyzed using Cytobank software.

In vitro induction of CRTAM expression on human NK cells. PBMCs, which were obtained from the Stanford Blood Center, were isolated from healthy donors by density gradient separation using Ficoll-Paque PLUS (Amersham Biosciences). NK cells were purified by negative magnetic cell sorting using NK cell isolation beads (Miltenyi Biotec). PBMCs and/or purified NK cells were cultured for 24 hours in complete medium alone, in medium containing control Rat IgG alone (10 μμg/mL), or with tumor cell line cells (at 1:1 PBMC or NK cell:tumor cell ratio) in the presence of Trastuzumab. Assessment of surface marker expression on NK cells was performed in triplicates for each condition.

In vitro NK cell cytotoxicity assays. PBMCs were incubated for 24 hours with irradiated (50 Gy) HER2-expressing breast cancer cells (HER18) at a ratio of 1:1 and with Trastuzumab (10 μg/ml). After 24 hours, NK cells were purified by negative magnetic cell sorting using NK cell isolation beads (Miltenyi Biotec) according to the manufacturer's instructions and to a greater than 90% purity, as defined by CD3-negative and⁻CD56-positive and confirmed by flow cytometry. Activation of NK cells was confirmed by flow cytometry. NK cell cytotoxicity was additionally measured by a chromium release assay: target cancer cells were labeled with 150 μCi ⁵¹Cr per 1×10⁶ cells for 2 hours, and subsequently added to activated PBMCs at variable effector/target cell ratios from 2:1 to 50:1. Percentage of cell lysis was determined after 4 hours of culture in the presence of the media (i.e., alone), anti-HER2 antibody (Trastuzumab, 10 μg/ml), agonistic anti-CRTAM antibody (IB4, 10 μg/ml), or combinations of this anti-HER2 antibody with an anti-CRTAM antibody (each at 10 μg/ml), with or without an agonistic anti-CD137 antibody (10 μg/ml). All assays were performed in triplicate with 3 independent NK cell samples.

Transplantation of breast cancer cells and antibody therapy. HER2-positive BT474M1 breast cancer cells were implanted subcutaneously into 5- to 6-week-old female athymic nu/nu mice at a dose of 5×10⁶ cells in 50 μl of PBS mixed with 50 μl of Matrigel (BD Biosciences) 1 day after sub-cutaneous implantation of a 0.72 mg/60 d release β-estradiol pellet (Innovative Research of America). After tumor inoculation, mice received by intraperitoneal (i.p.) injection control Rat IgG antibody (150 μg/injection), Trastuzumab (150 μg/injection), or agonistic antibody to CRTAM (150 μg/injection) on day 3, 10 and 17. The size of tumor mass was measured by caliper twice a week and expressed as the product of length by width in square centimeters. Mice were sacrificed when tumor size reached 4 cm² or when tumor sites ulcerated. All in vivo models were piloted with 5 mice per group.

### Results

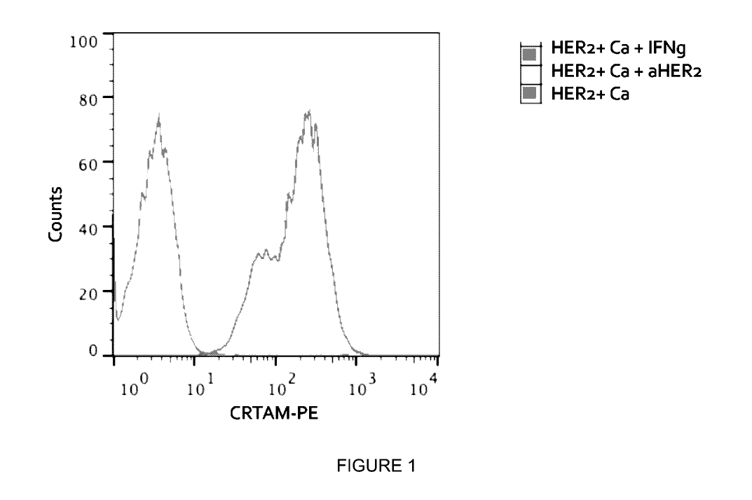

Expression of CRTAM on surfaces of PBMCs or NK cells was assessed in an in vitro model. Specifically, preparations of human PBMCs or human NK cells were grown in culture either alone or in the presence of cancer cell lines (HER2-positive breast cancer cells) that had been exposed to anti-cancer antigen antibody (Trastuzumab, which binds to HER2). The effect of antibody-coated cancer cells on CRTAM expression was measured by flow cytometry. Results are shown in FIG. 1. The data demonstrate that CRTAM expression level on immune effector cells can be enhanced by contact with tumor cells bound by an anti-cancer antigen antibody.

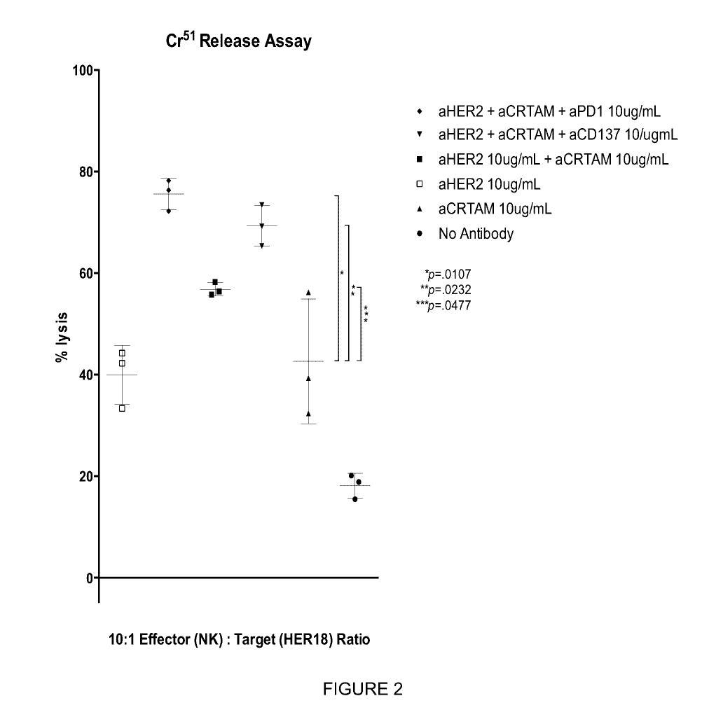

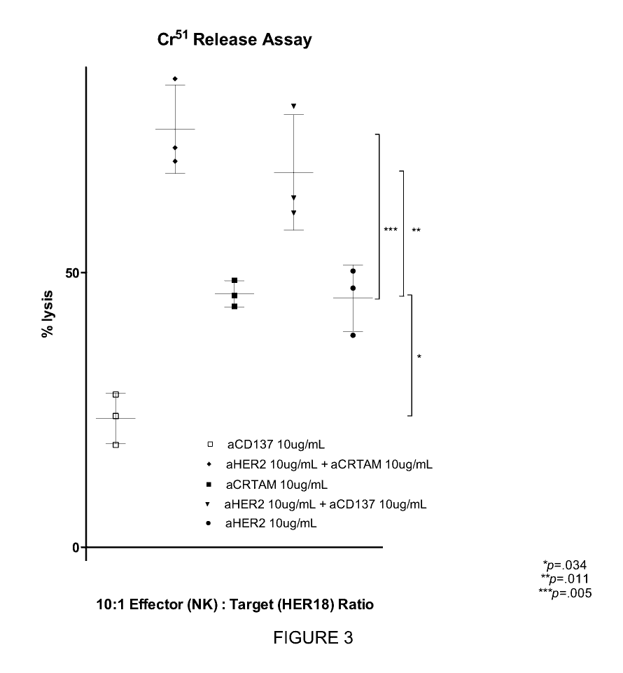

Activated PBMCs (containing CRTAM-positive NK cells) were incubated with target cells (HER18 breast cancer cells, which express HER2) in the presence of media alone (i.e., negative control), agonist anti-CRTAM antibody, anti-HER2 antibody (Trastuzumab), combinations of these anti-CRTAM and anti-HER2 antibodies, and a combination of anti-HER2 antibody, agonist anti-CRTAM antibody, and another agonist antibody targeting a different inducible effector cell surface marker (CD137). FIGS. 2-3 presents the results of these studies.

As can be seen, some cell lysis was observed in the presence of agonistic anti-CRTAM antibody. Similar levels of lysis were observed with anti-tumor antibody (i.e., anti-HER2) alone, but the level was dramatically amplified in the presence of agonistic anti-CRTAM antibody.

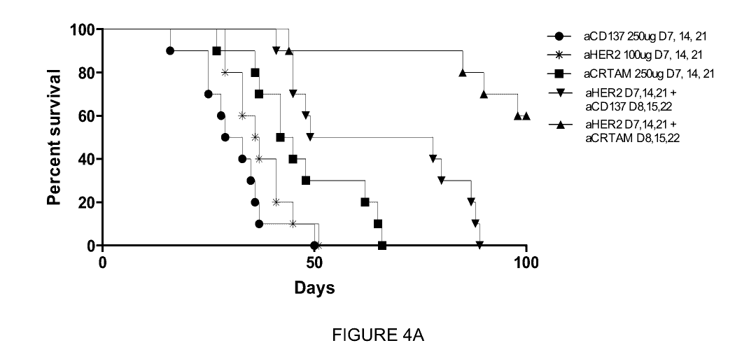

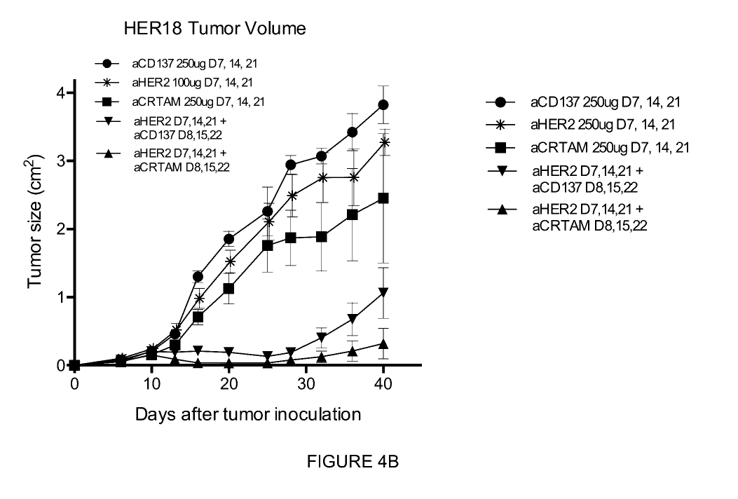

FIGS. 4 and 5 confirm that the in vitro findings hold true in an in vivo tumor model. Specifically, a HER2-expressing human cancer cell line was transferred in mice and the growth of tumor was measured in the following weeks during which alternative combinations treatments were tested. Regular administration of either an anti-tumor antibody (anti-HER2) or an agonistic antibody that targets an inducible effector cell surface marker (anti-CRTAM or anti-CD137) as monotherapy provided some reduction of growth of the tumor in mice. The therapeutic effect of anti-HER2 antibody (Trastuzumab) administration was dramatically enhanced by the administration of agonist antibody to anti-CRTAM or anti-CD137. These data confirm that agonizing CRTAM can effectively increase the ADCC activity of immune effector cells (e.g. NK cells) that is specifically directed at tumor cells (in particular at breast cancer cells), and that this increase is significantly enhanced when the tumor cells are bound by anti-tumor antibody.

Those skilled in the art will appreciate that the present invention is defined by the appended claims and not by the Examples or other description of certain embodiments included herein. For example, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, unless specifically excluded limit in the stated range. Where the stated range includes one or both limits, ranges excluding either or both of those limits are also included in the invention.

Similarly, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

Also, all publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

What is claimed is:

1. A method of treating cancer in a patient, the method comprising:
administering to the patient an effective dose of a CRTAM agonist.

2. The method of claim 1, wherein the CRTAM agonist is administered in combination with an anti-tumor antibody.

3. The method of claim 2, wherein the CRTAM agonist is administered a period of time after the anti-tumor antibody therapy, such that CRTAM expression has increased on immune effector cells.

4. The method of claim 3, further comprising at least one step of:
determining CRTAM expression level on the effector cells in a patient sample, the determining being performed prior to the step of administering the CRTAM agonist.

5. The method of claim 4, wherein the patient sample is a patient blood sample or cellular fraction thereof.

6. The method of any one of claims 4, wherein determining CRTAM expression level on surfaces of the effector cells comprises quantitating CRTAM protein.

7. The method of claim 3, wherein the period of time has a length between 1 hour and 5 days.

8. The method of claim 1, further comprising a second step of administering a second agonist, the second agonist being of a cell surface marker other than CRTAM, whose expression is increased on surfaces of immune effector cells when such cells are exposed to tumor cells bound by an anti-tumor antibody.

9. The method of claim 1, wherein the immune effector cells are CD3-negative CD56-positive NK cells.

10. The method of claim 2, wherein the anti-tumor antibody is a monoclonal antibody.

11. The method of claim 10, wherein the anti-tumor antibody is a humanized antibody.

12. The method of claim 10, wherein the anti-tumor antibody is a chimeric antibody.

13. The method of claim 1, wherein the cancer is a solid tumor.

14. The method of claim 13, wherein the solid tumor is a carcinoma.

15. The method of claim 13, wherein the solid tumor is a lymphoma.

16. The method of claim 1, wherein the cancer is a hematologic cancer.

17. A kit for use in the methods of claim 1.
